(19) United States
(12) Patent Application Publication
(10) Pub. No.: US 2018/0206732 A1
(43) Pub. Date: Jul. 26, 2018

(54) MEASUREMENT APPARATUS AND MEASUREMENT METHOD

(71) Applicant: SEIKO EPSON CORPORATION, Tokyo (JP)

(72) Inventor: Megumi ENARI, Suwa-shi (JP)

(73) Assignee: SEIKO EPSON CORPORATION, Tokyo (JP)

(21) Appl. No.: 15/865,619

(22) Filed: Jan. 9, 2018

(30) Foreign Application Priority Data
Jan. 26, 2017 (JP) 2017-012243

(51) Int. Cl. A61B 5/022; A61B 5/021

(52) U.S. Cl. CPC A61B 5/022; A61B 5/02141; A61B 5/02125

(57) ABSTRACT

A measurement apparatus includes a first pulse wave detection unit that detects a pulse wave at a first part pressed by a first pressing portion; a second pulse wave detection unit that detects the pulse wave at a second part pressed by a second pressing portion, the second part being located in a direction in which a traveling wave of the pulse wave travels from the first part; and an index measurement unit that measures an index relating to pulse wave propagation according to the pulse wave detected by the first pulse wave detection unit and the pulse wave detected by the second pulse wave detection unit, in which a pressing force first pressing portion exceeds a pressing force by the second pressing portion.

### BACKGROUND

### 1. Technical Field

The present invention relates to a technique for measuring biological information indicating a state of a living body.

### 2. Related Art

Various measurement techniques for measuring biological information such as a pulse wave propagation velocity have been proposed in the related art. For example, JP-A-2008-18035 discloses a measurement apparatus that measures a pulse wave propagation velocity by using a cuff attached to each of an upper limb and a lower limb of a living body. Specifically, the pulse wave propagation velocity of the living body is calculated using a time difference between the pulse wave detected by the cuff on the upper limb side and the pulse wave detected by the cuff on the lower limb side.

In the technique of JP-A-2008-18035, it is necessary to attach a cuff to each of the upper limb and the lower limb of the living body, so it is difficult to measure the pulse wave propagation velocity in daily life at all times, for example. On the other hand, if it is configured to detect pulse waves at two points close to each other, it is necessary to downsize the measurement apparatus. However, as the two points for detecting the pulse waves are closer to each other, for example, the influence of the pressure at which the cuff presses each point relatively increases, and as a result, there is a problem that the measurement accuracy of the pulse wave propagation velocity decreases.

### SUMMARY

An advantage of some aspects of the invention is to measure an index relating to pulse wave propagation with high accuracy even in a case where the two points for detecting pulse waves are close to each other.

A measurement apparatus according to a preferred aspect of the invention includes a first pulse wave detection unit that detects a pulse wave at a first part pressed by a first pressing portion; a second pulse wave detection unit that detects the pulse wave at a second part pressed by a second pressing portion, the second part being located in a direction in which a traveling wave of the pulse wave travels from the first part; and an index measurement unit that measures an index relating to pulse wave propagation according to the pulse wave detected by the first pulse wave detection unit and the pulse wave detected by the second pulse wave detection unit, in which a pressing force by the first pressing portion exceeds a pressing force by the second pressing portion. In the measurement apparatus, in the configuration of measuring the index relating to pulse wave propagation according to the pulse wave at the first part pressed by the first pressing portion and the pulse wave at the second part pressed by the second pressing portion, the pressing force by the first pressing portion exceeds the pressing force by the second pressing portion. Therefore, even in a case where the first part and the second part are close to each other, it is possible to measure the index relating to pulse wave propagation with high accuracy.

In a preferred aspect of the invention, the pressing force by the first pressing portion is 200 mmHg or less, and the pressing force by the second pressing portion is 80 mmHg or less. In a more preferred aspect of the invention, the pressing force by the first pressing portion 100 mmHg or less. According to the aspects, an effect that the index relating to pulse wave propagation can be measured with high accuracy is particularly remarkable.

In a preferred aspect of the invention, the first pulse wave detection unit includes a first pressure sensor that detects a pressure corresponding to displacement of the first part, and the second pulse wave detection unit includes a second pressure sensor that detects a pressure corresponding to displacement of the second part. According to the aspect, it is possible to specify the pressing force by the first pressing portion from the detection result by the first pulse wave detection unit and to specify the pressing force by the second pressing portion from the detection result by the second pulse wave detection unit.

In a preferred aspect of the invention, the measurement apparatus further includes a pressing force specifying unit that specifies the pressing force by the first pressing portion from a detection result by the first pulse wave detection unit and the pressing force by the second pressing portion from a detection result by the second pulse wave detection unit; and a determination processing unit that determines whether or not the pressing force by the first pressing portion and the pressing force by the second pressing portion, which are specified by the pressing force specifying unit, are appropriate. In the aspect, since the determination processing unit determines whether or not the pressing force by the first pressing portion and the pressing force by the second pressing portion are appropriate, there is an advantage that each pressing force can be easily adjusted to an appropriate range.

In a preferred aspect of the invention, the index measurement unit estimates a blood pressure from the index relating to the pulse wave propagation. In the aspect, there is an advantage that it is possible to estimate a blood pressure that is familiar to a large number of subjects.

A measurement method according to a preferred aspect of the invention includes detecting a pulse wave at a first part and a pulse wave at a second part, in a state where a pressing force pressing the first part of a measurement part exceeds a pressing force pressing the second part located in a direction in which a traveling wave of the pulse wave travels from the first part, of the measurement part; and measuring an index relating to pulse wave propagation according to the pulse wave at the first part and the pulse wave at the second part. In the aspect, in the measurement method of measuring the index relating to pulse wave propagation according to the pulse wave at the first part pressed by the first pressing portion and the pulse wave at the second part pressed by the second pressing portion, the pressing force by the first pressing portion exceeds the pressing force by the second pressing portion. Therefore, even in a case where the first part and the second part are close to each other, it is possible to measure the index relating to pulse wave propagation with high accuracy.

### BRIEF DESCRIPTION OF THE DRAWINGS

The invention will be described with reference to the accompanying drawings, wherein like numbers reference like elements.

FIG. 1 is a side view of a measurement apparatus according to a first embodiment of the invention.

FIG. 2 is a configuration diagram of the measurement apparatus.

FIG. 3 is a graph showing a relationship between a pushing amount of each of a first pressing portion and a second pressing portion and a pulse wave propagation velocity.

FIG. 4 is a graph showing a relationship between a pushing amount on an upstream side and the pulse wave propagation velocity in a case where a pushing amount on a downstream side of a traveling wave is fixed.

FIG. 5 is a graph showing a relationship between the pushing amount of the traveling wave on the downstream side and the pulse wave propagation velocity in a case where the pushing amount on the upstream side is fixed.

FIG. 6 is a graph showing a relationship between a pressing force of each of the first pressing portion and the second pressing portion and the pulse wave propagation velocity.

FIG. 7 is a graph showing an enlarged range in FIG. 6.

FIG. 8 is an explanatory diagram of a configuration for measuring the pressing force of each of the first pressing portion and the second pressing portion.

FIG. 9 is a side view of the measurement apparatus focused on a belt of the first embodiment.

FIG. 10 is an explanatory diagram of a process of specifying a pressing force from a first detection signal.

FIG. 11 is a flowchart of a measurement process.

FIG. 12 is a configuration diagram of a detection device of a second embodiment.

FIG. 13 is a flowchart of a measurement process in the second embodiment.

FIG. 14 is a configuration diagram of a detection device of a modification example.

FIG. 15 is a configuration diagram of a detection device of another modification example.

FIG. 16 is a configuration diagram of a detection device of still another modification example.

### DESCRIPTION OF EXEMPLARY EMBODIMENTS

### First Embodiment

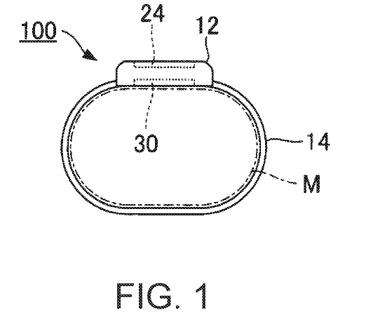

FIG. 1 is a configuration diagram of a measurement apparatus 100 according to a first embodiment of the invention. The measurement apparatus 100 of the first embodiment is a living body measurement device that non-invasively measures the biological information of a subject (an example of a living body), and is worn on a site to be measured (hereinafter referred to as “measurement part”) of the subject's body. As illustrated in FIG. 1, the measurement apparatus 100 of the first embodiment is a wristwatch-type portable device having a housing portion 12 and a belt 14, and is worn on the subject's body by winding the belt 14 of a band shape on a wrist (or a forearm) which is an example of the measurement part M. In the first embodiment, the pulse wave propagation velocity (PWV: Pulse Wave Velocity) is exemplified as biological information. The pulse wave propagation velocity is a velocity at which the pulse wave generated by the beat of the heart propagates in the artery, and is suitably used for diagnosis of diseases such arteriosclerosis as the index reflecting the hardness of the artery.

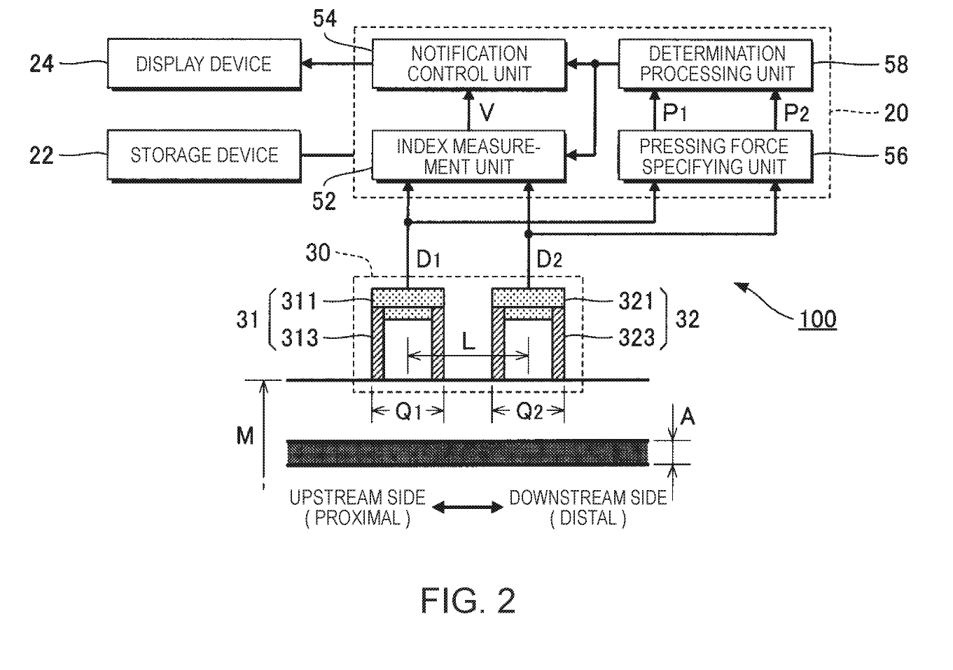

FIG. 2 is a configuration diagram focused on the function of the measurement apparatus 100. As illustrated in FIG. 2, the measurement apparatus 100 of the first embodiment includes a controller 20, a storage device 22, a display device 24, and a detection device 30. The controller 20 and the storage device 22 are provided inside the housing portion 12. As illustrated in FIG. 1, the display device 24 (for example, a liquid crystal display panel) is provided on the surface opposite to the surface facing the measurement part M in the housing portion 12, and displays various types of images including the measurement result under the control of the controller 20.

The detection device 30 of FIG. 2 is a sensor module that generates a first detection signal D1 and a second detection signal D2 depending on the state of the measurement part M, and is provided, fear example, on the part facing the measurement par the housing portion 12. Each of the first detection signal D1 and the second detection signal. D2 is a signal representing a pulse wave that propagates an artery A (for example, radial artery or ulnar artery) inside the measurement part M. As illustrated in FIG. 2, the detection device 30 of the first embodiment includes a first pulse wave detection unit 31 and a second pulse wave detection unit 32.

The first pulse wave detection unit 31 is a sensor including a portion 313 (hereinafter referred to as “first pressing portion”) which presses the measurement part M, and detects a pulse wave (hereinafter referred to as “pulse wave in the first part Q1”) that travels the artery A in a part Q1 (hereinafter referred to as “first part”) of the measurement part M pressed by the first pressing portion 313 to generate a first detection signal D1. That is, the first detection signal D1 is a signal representing the beat of the artery A at the first part Q1.

Specifically, as illustrated in FIG. 2, the first pulse wave detection unit 31 includes a first pressure sensor 311 and a first pressing portion 313. The first pressing portion 313 is a tubular body made of an elastic material (for example, a tube having a circular cross section). The first pressure sensor 311 is configured with, for example, an air pressure sensor (for example, a sensor IC configured with a semiconductor integrated circuit) or a gauge pressure sensor, and is provided inside one end of the first pressing portion 313. The end surface of the first pressing portion 313 on the side opposite to the first pressure sensor 311 is pressed against the surface of the measurement part M, and the space inside the tube (hereinafter, referred to as “measurement space”) is sealed. When the surface of the measurement part M is displaced due to the beat of the artery A at the first part Q1 in the measurement part, the volume of the measurement space fluctuates, so the pressure in the measurement space periodically fluctuates in conjunction with the beat of the artery A inside the first part Q1. The first pressure sensor 311 generates a first detection signal D1 indicating the pressure in the measurement space (that is, the pressure corresponding to the displacement of the first part Q1). As understood from the above description, the first detection signal D1 is a pulse wave signal including a periodic fluctuation component corresponding to the beat component (pressure pulse wave) of the artery A inside the first part Q1. That is, the first pulse wave detection unit 31 functions as an element that detects a pulse wave at the first part Q1 of the subject.

Like the first pulse wave detection unit 31, the second pulse wave detection unit 32 is a sensor including a part 323 that presses the measurement part M (hereinafter referred to as “second pressing portion”). The second pulse wave detection unit 32 detects a pulse wave (hereinafter referred to as “pulse wave in the second part Q2”) that travels the artery A in a part Q2 (hereinafter referred to as “second part”) of the measurement part M pressed by the second pressing portion 323, and generates a second detection signal D2. That is, the second detection signal D2 is a signal representing the beat of the artery A in the second part Q2.

Specifically, the second pulse wave detection unit 32 is configured to include a second pressure sensor 321 and a second pressing portion 323, similarly to the first pulse wave detection unit 31. The second pressing portion 323 is a tubular body made of an elastic material and having a circular cross section. The pressure in the measurement space inside the second pressing portion 323 periodically fluctuates in conjunction with the beat of the artery A of the second part Q2. The second pressure sensor 321 is configured with an air pressure sensor or a gauge pressure sensor provided inside one end of the second pressing portion 323. Similar to the first pulse wave detection unit 31, the second pressure sensor 321 of the second pulse wave detection unit 32 generates a second detection signal D2 indicating the pressure in the measurement space of the second pressing portion 323 (that is, the pressure corresponding to the displacement of the second part Q2). As understood from the above description, the second detection signal D2 is a pulse wave signal which periodically fluctuates in conjunction with the beat of the artery A in the second part Q2. That is, the second pulse wave detection unit 32 functions as an element that detects a pulse wave at the second part Q2 of the subject. The illustration of an A/D converter that converts the first detection signal D1 generated by the first pulse wave detection unit 31 and the second detection signal D2 generated by the second pulse wave detection unit 32 from analog to digital is omitted for the sake of convenience.

As illustrated in FIG. 2, the first pressing portion 313 and the second pressing portion 323 are spaced apart from each other along the artery A inside the measurement part M. Specifically, the second pressing portion 323 is located on the distal side (the side opposite to the heart) from the first pressing portion 313. In other words, the second part Q2 of the measurement part M is located on the side to which the traveling wave of the pulse wave travels the artery A from the first part Q1 (that is, downstream side of the traveling wave). Therefore, the pulse wave in the first part Q1 is delayed by the time spending to the distance L between the first pressing portion 313 and the second pressing portion 323 to become the pulse wave in the second part Q2. The distance L is, for example, a distance between the centers of the first pressing portion 313 and the second pressing portion 323.

The controller 20 shown in FIG. 2 is an arithmetic processing device such as a central processing unit (CPU) or a field-programmable gate array (FPGA), and controls the entire measurement apparatus 100. The storage device 22 is configured with, for example, a nonvolatile semiconductor memory, and stores programs executed by the controller 20 and various data used by the controller 20. Although the controller 20 and the storage device 22 are illustrated as separate elements in FIG. 2, the controller 20 including the storage device 22 may be realized by, for example, an application specific integrated circuit (ASIC) or the like.

The controller 20 of the first embodiment executes the program stored in the storage device 22 to realize a plurality of functions regarding the measurement of the pulse wave propagation velocity V of a subject (the index measurement unit 52, the notification control unit 54, the pressing force specifying unit 56, and the determination processing unit 58). A configuration in which the functions of the controller 20 are distributed to a plurality of integrated circuits or a configuration in which a part or all of the functions of the controller 20 is realized by dedicated electronic circuits can be adopted.

The index measurement unit 2 measures a pulse wave propagation velocity V, according to the pulse wave (first detection signal D1) detected by the first pulse wave detection unit 31 from the first part Q1 and the pulse wave (second detection signal D2) detected by the second pulse wave detection unit 32 from the second part Q2. Specifically, the index measurement unit 52 calculates the pulse wave propagation velocity V (V=L/Δt) by dividing the distance L between the first pressing portion 313 and the second pressing portion 323 by the time difference Δt between the rising times of the first detection signal D1 and the second detection signal D2. Actually, a deterministic pulse wave propagation velocity V is calculated by averaging the pulse wave propagation velocity V calculated every beat of pulse wave over multiple beats (for example, 10 beats). The rising time of each of the first detection signal D1 and the second detection signal D2 is, for example, the time at which the signal value becomes the minimum value, the time at which the first derivative value of the signal value becomes the maximum, or the second derivative value of the signal value becomes maximum. In the first embodiment, the time at which the first derivative value of the signal value becomes maximum is set as the rising time.

The notification control unit 54 in FIG. 2 displays the measurement result (specifically, the pulse wave propagation velocity V) by the index measurement unit 52 on the display device 24. It is also possible for the notification control unit 54 to display on the display device 24 whether or not the pulse wave propagation velocity V is the numerical value within a normal range (that is, presence or absence of abnormality of the subject). It is also possible to notify the subject of the numerical value of the pulse wave propagate on velocity V and the presence or absence of abnormality by voice.

However, the pulse wave propagation velocity V tends to depend on the pressure (hereinafter referred to as “pressing force”) at which the measurement part M is pressed at the time of measurement. Specifically, the tendency is observed that the measurement value of the pulse wave propagation velocity V becomes a smaller numerical value as the pressing force for the measurement part M is larger. On the assumption of the above tendency, from the viewpoint of measuring the pulse wave propagation velocity V with high accuracy, the inventor of the invention examined the condition of the pressing force capable of properly measuring the pulse wave propagation velocity V.

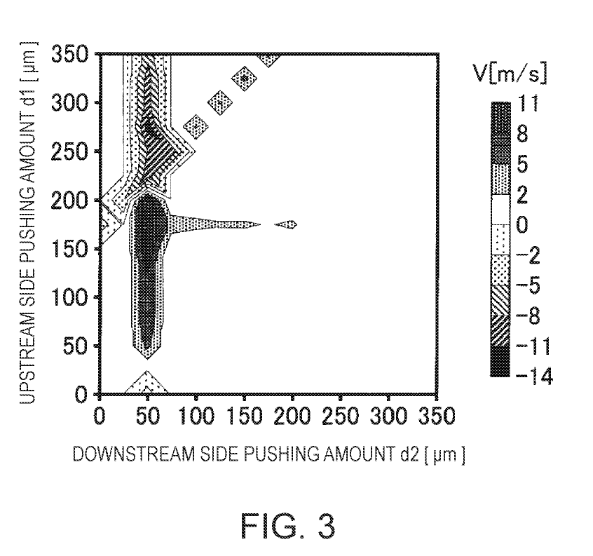

FIG. 3 shows the distribution of the pulse wave propagation velocity V measured in each case where each of the first pressing portion 313 and the second pressing portion 323 are displaced. The measured values in FIG. 3 are measured with a distance L for the part near the subject's radial artery as of 46 mm. The pushing amount d1 of the first pressing portion 313 with respect to the measurement part M is shown on the vertical axis and the pushing amount d2 of the second pressing portion 323 with respect to the measurement part M is shown on the horizontal axis. The micrometer is fixed to each of the first pulse wave detection unit 31 and the second pulse wave detection unit 32, and the pushing amount d1 and the pushing amount d2 are individually controlled. In the test of observing the relationship among the pushing amount d1, the pushing amount d2, and the pulse wave propagation velocity V, a healthy subject, whose pulse wave propagation velocity V is within the normal range, is maintained in the sitting posture, and the detection device 30 is gradually moved close to the surface of the measurement part M. The position of the first pressing portion 313 when the first pulse wave detection unit 31 starts to detect the pulse wave is the origin (d1=0) of the pushing amount d1, and the position of the second pressing portion 323 when the second pulse wave detection unit 32 starts to detect the pulse wave is the origin (d2=0) of the pushing amount d2. FIG. is a graph showing a relationship between the pushing amount d1 on the upstream side and the pulse wave propagation velocity V in a state where the pushing amount d2 on the downstream side of the traveling wave is fixed at 50 μm. FIG. 5 is a graph showing a relationship between the pushing amount d2 on the downstream side of the traveling wave and the pulse wave propagation velocity V in a state where the pushing amount dl on the upstream side is fixed at 175 μm.

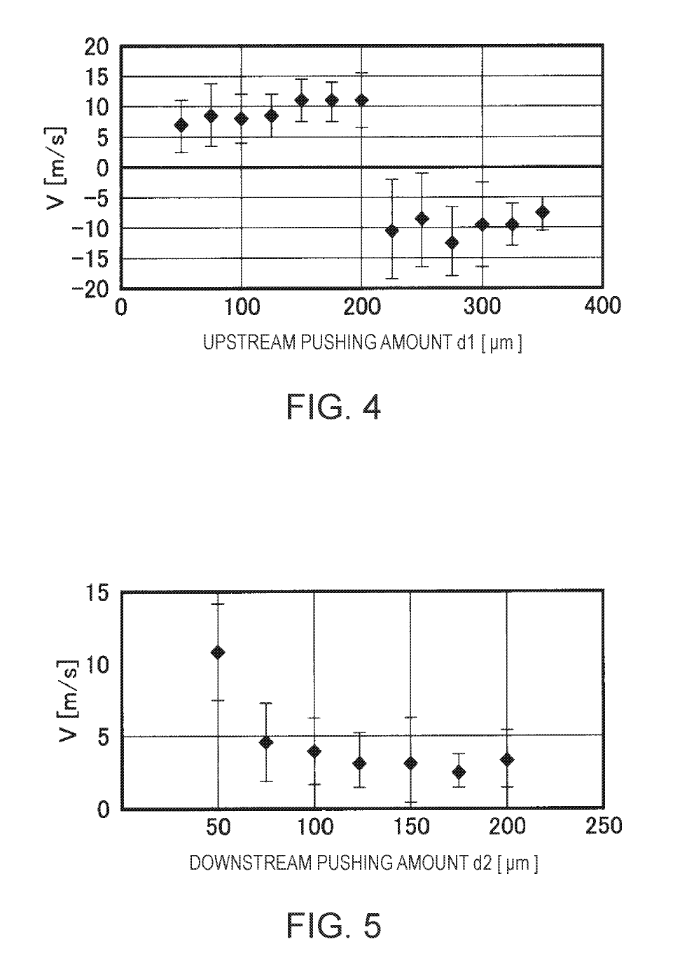

The normal range of the pulse wave propagation velocity V in the radial artery is roughly in the range of 8 m/s or more and 12 m/s or less. Since the tests of FIGS. 3 to 5 are performed on the healthy subjects confirmed by the prior diagnosis in which the pulse wave propagation velocity V is within the normal range, if the pulse wave propagation velocity V is within the range of 8 m/s or more and 12 m/s or less, it can be determined that the pulse wave propagation velocity V has been properly measured. As can be seen from FIGS. 3 to 5, in a case where the pushing amount d1 of the first pressing portion 313 is within a range from 150 μm to 200 μm and the pushing amount d2 of the second pressing portion 323 is a numerical value around 50 atm, the pulse wave propagation velocity V is measured properly. As understood from the results of the test described above, in order to appropriately measure the pulse wave propagation velocity V from the pulse wave at the first part Q1 and the pulse wave at the second part Q2, it is necessary to make the pushing amount d1 of the first pressing portion 313 and the pushing amount d2 of the second pressing portion 323 different from each other.

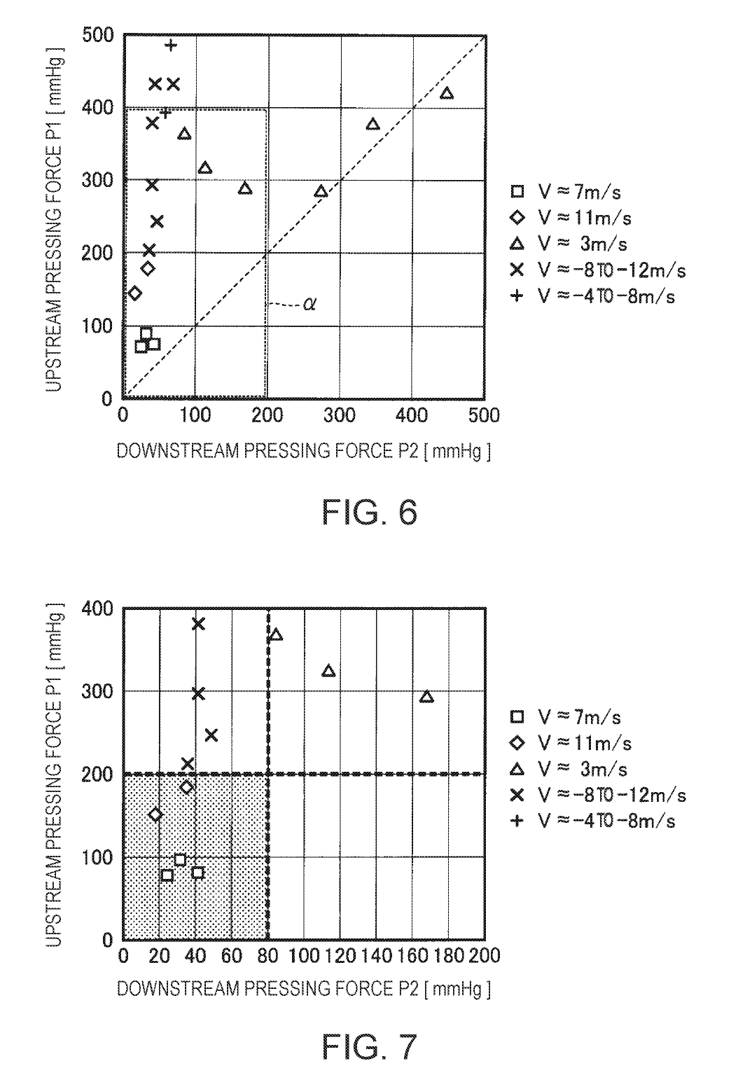

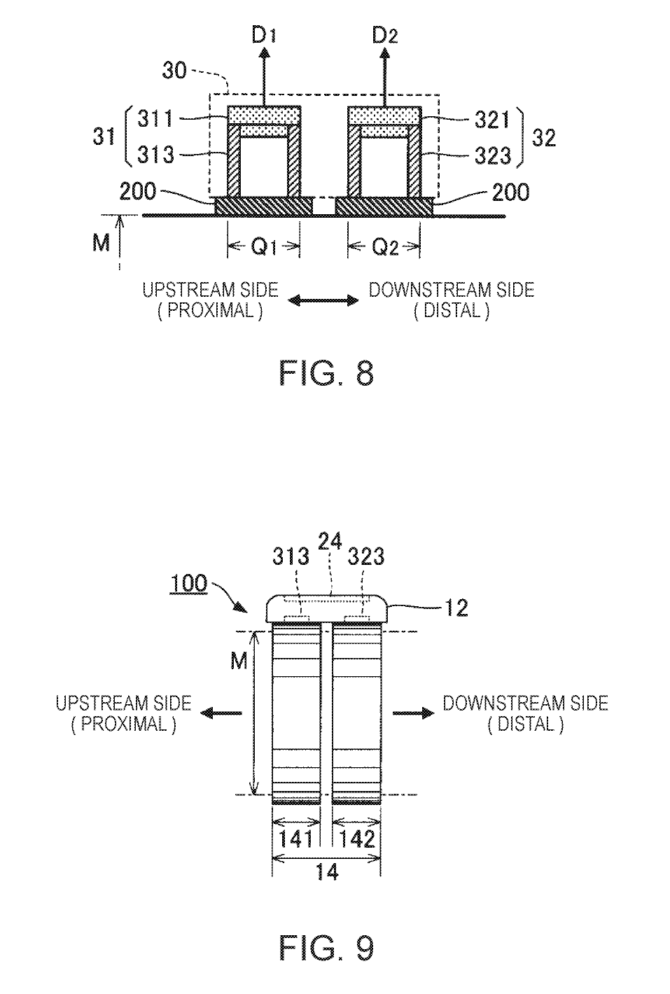

The pushing amount d1 corresponds to the pressing force P1 by which the first pressing portion 313 presses the first part Q1 of the measurement part M. The pushing amount d2 corresponds to the pressing force P2 by which the second pressing portion 323 presses the second part Q2 of the measurement part M. FIG. 6 is a graph showing the relationship among the pressing force P1 by the first pressing portion 313, the pressing force P2 by the second pressing portion 323, and the pulse wave propagation velocity V, based on the results of the tests of FIGS. 3 to 5, and FIG. 7 is a graph showing an enlarged range a in FIG. 6. Further, a relationship between the pressing forces (P1, P2) detected by the pressure sensor 200 provided between each of the first pressing portion 313 and the second pressing portion 323, as illustrated in FIG. 8 and the measurement part N and the pulse wave propagation velocity V calculated from the first detection signal D1 and the second detection signal D2 is shown in FIG. 6 and FIG. 7. The unit of each of the pressing force P1 and the pressing force P2 is millimeter of mercury (mmHg).

As described with reference to FIG. 3, it can also be checked from FIG. 6 and FIG. 7 that it is necessary to make the pressing force P1 of the first pressing portion 313 with respect to the first part Q1 different from the pressing force P2 of the second pressing portion 323 with respect to the second part Q2. Specifically, in a case where the pulse wave propagation velocity V is properly measured (V≅7 m/s, 11 m/s), the ranges of the pressing force P1 and the pressing force P2 are within the range where the pressing force P1 exceeds the Dressing force P2. Given the background of the above findings, in the first embodiment, the pulse wave propagation velocity V is measured in a state (P1>P2) where the pressing force P1 by the first pressing portion 313 exceeds the pressing force P2 by the second pressing portion 323. In the following description, the fact that the pressing force P1 exceeds the pressing force P2 is expressed as “first condition”.

For example, in the vicinity of the wrist (for example, in the vicinity of the distal end of the radius), the artery A exists at a position deeper with respect to the skin surface, on the upstream side of the pulse wave. Therefore, in order to detect the beat of the artery A, it is necessary to push the measurement part M stronger, the upstream side of the pulse wave. It is assumed that the reason why the pressing force P1 needs to exceed the pressing force P2 in order to properly measure the pulse wave propagation velocity V, as described above, is that the artery A exists at a position deeper with respect to the skin surface, on the upstream side of the pulse wave.

As described above, in order to properly measure the pulse wave propagation velocity V, the pressing force P1 needs to exceed the pressing force P2. However, even in a case where the pressing force P1 exceeds the pressing force P2, there is a possibility that the pulse wave propagation velocity V cannot be measured properly in a state where each of the pressing force P1 and the pressing force P2 is excessively high. Specifically, as understood from FIG. 6 and FIG. 7, case where the pressing force P1 exceeds 200 mmHg or the pressing force P2 exceeds 80 mmHg, there is a possibility that pulse wave propagation velocity V cannot be properly measured. Considering the above circumstances, in the first embodiment, in the state where the pressing force P1 by the first pressing portion 313 is 200 mmHg or less and the pressing force P2 by the second pressing portion 323 is 80 mmHg or less, the pulse wave propagation velocity V is measured. In the following description, a fact that the second condition is that the pressing P1 is 200 mmHg or less and the pressing force P2 is 80 mmHg or less is described as “second condition”.

In the first embodiment, the pressing, force P1 by the first pressing portion 313 and the pressing force P2 by the second pressing portion 323 can be adjusted by the subject (or a user other than the subject). Specifically, by adjusting the belt 14 wound around the wrist of the subject, the subject can individually adjust the pressing force P1 and the pressing force P2. FIG. 9 is a side view of the measurement apparatus 100 focused on the configuration of the belt 14 in the first embodiment. FIG. 1 is a side view of the measurement apparatus 100 as viewed from the direction in which the artery A of the measurement part M extends, and FIG. 9 is a side view of the measurement apparatus 100 as viewed from a direction perpendicular to the direction in which the artery A extends.

As illustrated in FIG. 9, the belt 14 of the first embodiment includes a first attachment portion 141 and a second attachment portion 142. Each of the first attachment portion 141 and the second attachment portion 142 is a variable-length belt-like member (that is, a belt) wound around the wrist of the subject. The subject can individually adjust the tightening condition of the measurement part M by each of the first attachment portion 141 and the second attachment portion 142, by changing the total length (that is, the diameter) of each of the first attachment portion 141 and the second attachment portion 142.

As illustrated in FIG. 9, on the part facing the measurement part M in the housing portion 12, a first pressing portion 313 is provided within a range of the width of the first attachment portion 141 and a second pressing portion 323 is provided within a range of the width of the second attachment portion 142. Therefore, the pressing force P1 by the first pressing portion 313 is changed by adjusting the tightening condition of the first attachment portion 141, and the pressing force P2 by the second pressing portion 323 is changed by adjusting the tightening condition of the second attachment portion 142. Specifically, the pressing force P1 increases as the first attachment portion 141 contracts to strengthen the fastening, and the pressing force P2 increases as the second attachment portion 142 contracts to strengthen the fastening.

As mentioned above, the subject can adjust the pressing force P1 and the pressing force P2. In the first embodiment, the subject adjusts the pressing force P1 and the pressing force P2 such that the pressing force P1 and the pressing force P2 satisfy the first condition (P1>P)and the second condition (P1≤200 mmHg, P2≤80 mmHg). However, in a case where the measurement apparatus 100 is actually used, it is assumed that it is difficult for the subject to determine whether the pressing force P1 and the pressing force P2 are within an appropriate range. In view of the above circumstances, in the first embodiment, the controller 20 (the pressing force specifying unit 56 and the determination processing unit 58) determines whether or not the pressing force P1 by the first pressing portion 313 and the pressing force P2 by the second pressing portion 323 are appropriate.

The pressing force specifying unit 56 of FIG. 2 specifies the pressing force P1 by the first pressing portion 313 and the pressing force P2 by the second pressing portion 323. Specifically, the pressing force specifying unit 56 of the first embodiment specifies the pressing force P1 from the detection result (that is, the first detection signal D1) by the first pulse wave detection unit 31, and specifies the pressing force P2 from the detection result (that is, the second detection signal D2) by the second pulse wave detection unit 32.

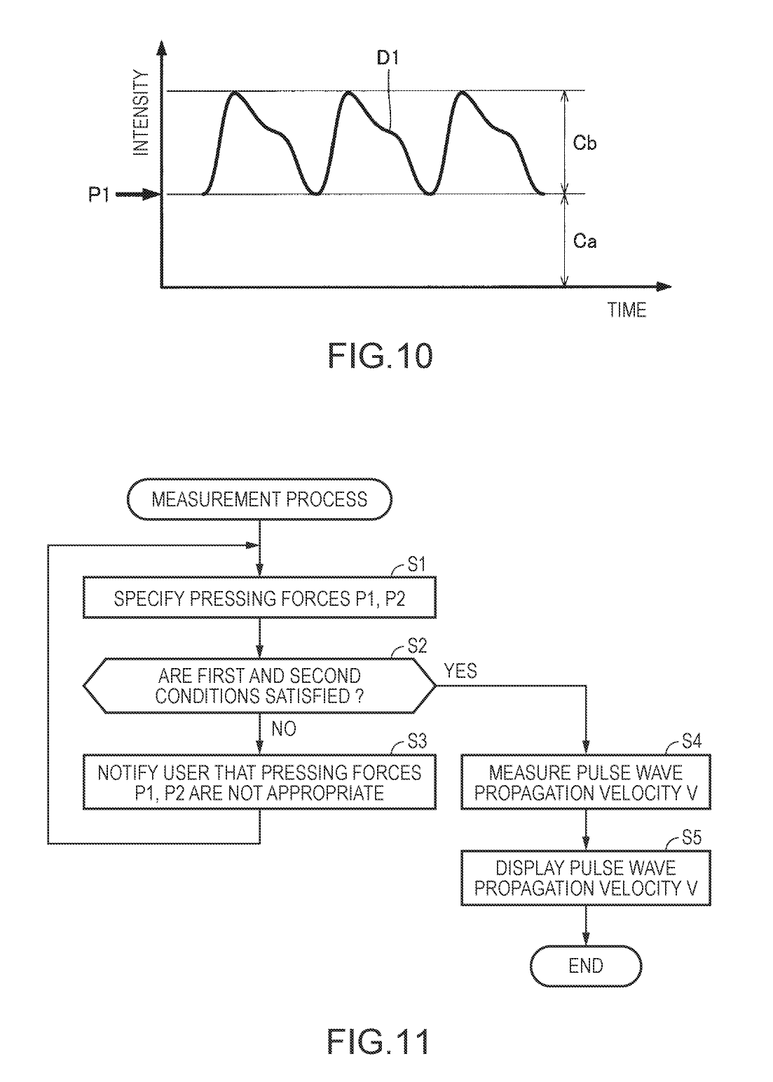

In FIG. 10, the waveform of the first detection signal D1 is illustrated. As illustrated in FIG. 10, the first detection signal D1 contains a stationary component Ca and a fluctuation component Cb. The stationary component Ca is a stationary signal component caused by the static pressing of the first pressing portion 313 on the first part Q1 and corresponds to a low frequency component (ideally a direct current component) whose frequency is lower than a predetermined threshold value. The fluctuation component Cb is a signal component periodically fluctuating due to the beat of the artery A inside the first part Q1, and corresponds to a high frequency component whose frequency exceeds the threshold value. The pressing force specifying unit 56 of the first embodiment specifies the pressing force P1 by the first pressing portion 313 according to the signal intensity of the stationary component Ca of the first detection signal D1. Specifically, the pressing force specifying unit 56 extracts the stationary component Ca by low-pass filtering on the first detection signal D1, and converts the average signal intensity of the stationary component Ca into the pressing force P1 by the first pressing portion 313. For calculation of the pressing force P1, a predetermined arithmetic expression that defines the relationship between the signal intensity of the stationary component Ca and the pressing force P1 is used. The pressing force specifying unit 56 specifies the pressing force P2 by the second pressing portion 323 from the stationary component Ca of the second detection signal D2 generated by the second pulse wave detection unit 32, similarly to the process for the first detection signal D1.

The determination processing unit 58 of FIG. 2 determines whether or not the pressing force P1 and the pressing force P2 specified by the pressing force specifying unit 56 are appropriate. Specifically, the determination processing unit 58 determines whether or not the first condition and the second condition are satisfied for the pressing force P1 and the pressing force P2. In a case where both the first condition and the second condition are satisfied, the pulse wave propagation velocity V is measured by the index measurement unit 52. On the other hand, in a case where one or both of the first condition and the second condition is not satisfied, the notification control unit 54 notifies the subject that the pressing force P1 and the pressing force P2 is inappropriate. For example, the notification control unit 54 displays an image (for example, a message such as “Please adjust the length of the belt”) instructing readjustment of the pressing force P1 and the pressing force P2, on the display device 24.

FIG. 11 is a flowchart of a process (hereinafter referred to as “measurement process”) executed by the controller 20 of the first embodiment. The measurement process of FIG. 11 is started in a case where the subject instructs the measurement of, for example, the pulse wave propagation velocity V.

When the measurement process is started, the pressing force specifying unit 56 specifies the pressing force P1 by the first pressing portion 313 and the pressing force P2 by the second pressing portion 323 (S1). Specifically, the pressing force specifying unit 56 specifies the pressing force P1 from the first detection signal D1 generated by the first pulse wave detection unit 31, and specifies the pressing force P2 from the second detection signal D2 generated by the second pulse wave detection unit 32. The determination processing unit 58 determines whether or not the pressing force P1 and the pressing force P2 specified by the pressing force specifying unit 56 satisfy the first condition (P1>P2) and the second condition (P1≤200 mmHg, P2≤80 mmHg) (S2).

In a case where either or both of the first condition and the second condition are not satisfied (S2: NO), the notification control unit 54 notifies the subject that the pressing force P1 and the pressing force P2 are not appropriate (S3) Specifically, an image instructing readjustment of the pressing force P1 and the pressing force P2 is displayed on the display device 24. When checking that pressing force P1 and the pressing force P2 are not appropriate by viewing the image on the display device 24, the subject changes the pressing force P1 and the pressing force P2 by adjusting each of the first attachment portion 141 and the second attachment portion 142. On the other hand, after the notification control unit 54 executes the notification, the process proceeds to step S1. That is, until the pressing force P1 and the pressing force P2 satisfy the first condition and the second condition by the adjustment by the subject, the specification (S1) of the pressing force P1 and the pressing force P2 and the determination (S2) of the success or failure of the first condition and the second condition are repeated.

As a result of adjustment by the subject, if the first condition and the second condition are satisfied (S2: YES), the index measurement unit measures the pulse wave propagation velocity V (S4). Specifically, the index measurement unit 52 measures a pulse wave propagation velocity V, according to the pulse wave (first detection signal D1) detected by the first pulse wave detection unit 31 from the first part Q1 and the pulse wave (second detection signal D2) detected by the second pulse wave detection unit 32 from the second part Q2. That is, by using the first detection signal D1 and the second detection signal D2 generated under the situation that the pressing force P1 and the pressing force P2 satisfy the first condition and the second condition, the pulse wave propagation velocity V of the measurement part M is measured. The notification control unit 54 displays the pulse wave propagation velocity V measured by the index measurement unit 52 on the display device 24 (S5).

As described above, in the first embodiment, the configuration of measuring the pulse wave propagation velocity V according to the pulse wave at the first part Q1 pressed by the first pressing portion 313 and the pulse wave at the second part Q2 pressed by the second pressing portion 323, the pressing force P1 exceeds the pressing force P2, Therefore, even in a case where the first part Q1 and the second part Q2 are close to each other, it is possible to measure the pulse wave propagation velocity V with high accuracy. In the first embodiment, in particular, in the state where the pressing force P1 by the first pressing portion 313 is 200 mmHg or less and the pressing force P2 by the second pressing portion 323 is 80 mmHg or less, the pulse wave propagation velocity V is measured. Therefore, the effect that the pulse wave propagation velocity V can be measured with high accuracy is particularly remarkable.

### Second Embodiment

A second embodiment of the invention will be described. In each of the following examples, the same reference numerals used in the description of the first embodiment are used for the elements whose actions or functions are the same as those of the first embodiment, and the detailed explanation thereof is appropriately omitted.

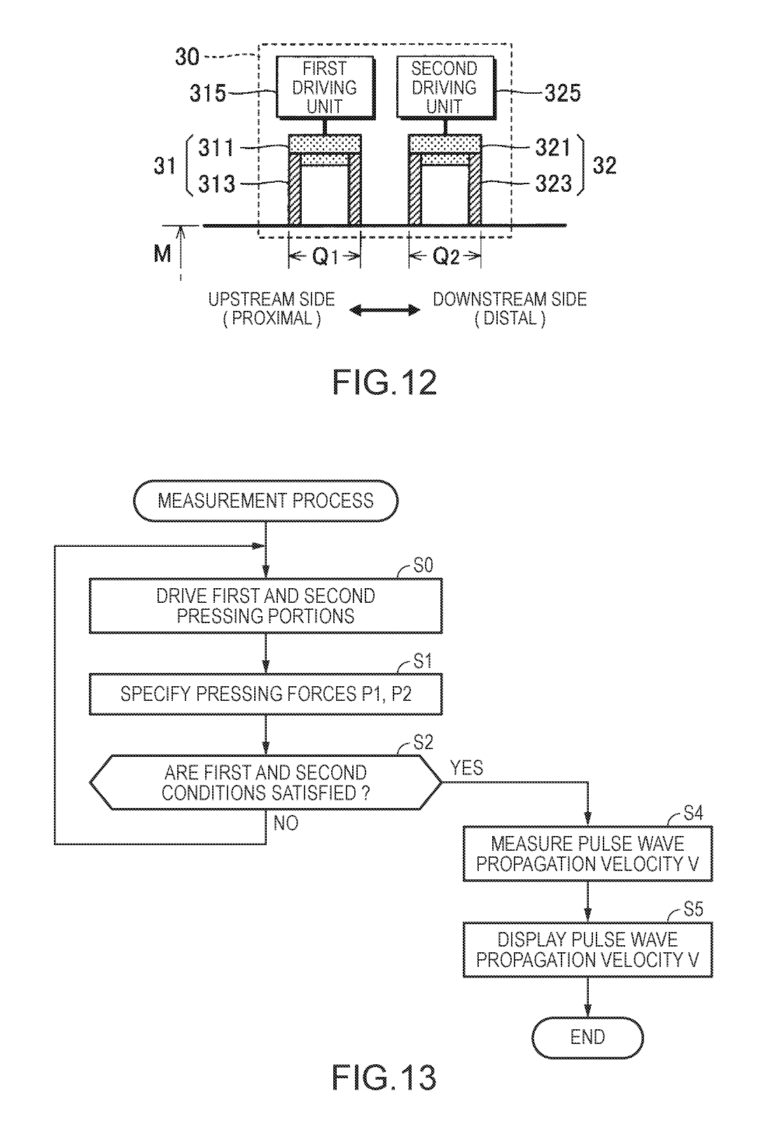

FIG. 12 is a configuration diagram of a detection device 30 of a second embodiment. As illustrated in FIG. 12, the detection device 30 of the second embodiment includes a first driving unit 315 and a second driving unit 325, in addition to the same first pulse wave detection unit 31 and the same second pulse wave detection unit 32 as those in the first embodiment. The first driving unit 315 displaces the first pressing portion 313 of the first pulse wave detection unit 31 under the control of the controller 20. The second driving unit 325 displaces the second pressing portion 323 of the second pulse wave detection unit 32 under the control of the controller 20. For example, an actuating mechanism (actuator) that displaces the first pressing portion 313 or the second pressing portion 323 by using a solenoid is suitably used as the first driving unit 315 and the second driving, unit 325. It is also possible to use an actuating mechanism that displaces the first pressing portion 313 or the second pressing portion 323 by using an air bag (cuff) which expands or contracts by suction and exhaust as the first driving unit 315 and the second driving unit 325.

FIG. 13 is a flowchart of the measurement process in the second embodiment. When the measurement process of FIG. 13 is started, the controller 20 displaces the first pressing portion 313 by driving the first driving unit 315, and displaces the second pressing portion 323 by driving the second driving unit 325 (S0). The pressing force P1 and the pressing force P2 change according to the operation of the first driving unit 315 and the second driving unit 325. The pressing force specifying unit 56 specifies the pressing force P1 and the pressing force P2 after the change in the same way as in the first embodiment (S1). The determination processing unit 58 determines whether or not. the pressing force P1 and the pressing force P2 specified by the pressing force specifying unit 56 satisfy the first condition (P1>P2) and the second condition. (P1≤200 mmHg, P2≤80 mmHg) (S2).

In a case where one or both of the first condition and the second condition are not satisfied (S2: NO), the process proceeds to step S0. That is, the controller 20 further displaces the first pressing portion 313 by driving the first driving unit 315, and further displaces the second pressing portion 343 by driving the second driving unit 325. That is, until the pressing force P1 and the pressing force P2 satisfy the first condition and the second condition, while the first pressing portion 313 and the second pressing portion 323 are stepwise displaced (S0), the specification (S1) of the pressing force P1 and the pressing force P2 and the determination (S2) of the success or failure of the first condition and the second condition are repeated. If the first condition and the second condition are satisfied as a result of the adjustment of the displacement amount of the first pressing portion 313 and the second pressing portion 323 (S2: YES), measurement (S4) and the display (S5) of pulse wave propagation velocity V are executed.

In the second embodiment, the same effect as the first embodiment is also realized. In the second embodiment, since the pressing force P1 and the pressing force P2 are controlled under the control of the first driving unit 315 and the second driving unit 325 by the controller 20, there is an advantage that the subject does not need to manually adjust the pressing force P1 and the pressing force P2. On the other hand, since the first driving unit 315 and the second driving unit 325 exemplified in the second embodiment is unnecessary in the first embodiment, there is an advantage that the configuration of the measurement apparatus 100 is simplified.

### Third Embodiment

In the first embodiment and the second embodiment, a configuration for measuring the pulse wave propagation velocity V is exemplified. In the third embodiment, the blood pressure of the subject is estimated. Note that the same configuration as the first embodiment or the second embodiment is adopted for specifying and adjusting the pressing force P1 and the pressing force P2.

The index measurement unit 52 of the third embodiment measures the pulse wave propagation velocity V from the first detection signal D1 and the second detection signal D2, and estimates the blood pressure (at least one of systolic blood pressure and diastolic blood pressure) of the subject from the pulse wave propagation velocity V, by the same method as the first embodiment or the second embodiment. Specifically, the index measurement unit 52 calculates the blood pressure, by applying the pulse wave propagation velocity V to the expression expressing the correlation between the numerical value of the pulse wave propagation velocity V and the numerical value of the blood pressure. It is also possible for the index measurement unit 52 to specify the blood pressure corresponding to the pulse wave propagation velocity V by referring to the table in which the correspondence between the numerical value of the pulse wave propagation velocity V and the numerical value of the blood pressure is registered.

The notification control unit 54 displays the blood pressure calculated by the index measurement unit 52 on the display device 24. It is also possible for the notification control unit 54 to display on the display device 24 whether or not the blood pressure is the numerical value within a normal range (that is, presence or absence of abnormality of the subject). It is also possible to notify the subject of the numerical value of blood pressure and the presence or absence of abnormality by voice.

In the third embodiment, the same effect as the first embodiment is also realized. Further, in the third embodiment, there is an advantage that it is possible to measure blood pressure that is more familiar than the pulse wave propagation velocity V for many subjects.

### MODIFICATION EXAMPLE

Each embodiment exemplified above can be variously modified. Specific modification aspects that can be applied to each of the above-described embodiments are exemplified below. Two or more aspects arbitrarily selected from the following examples can be appropriately combined within a range not inconsistent with each other.

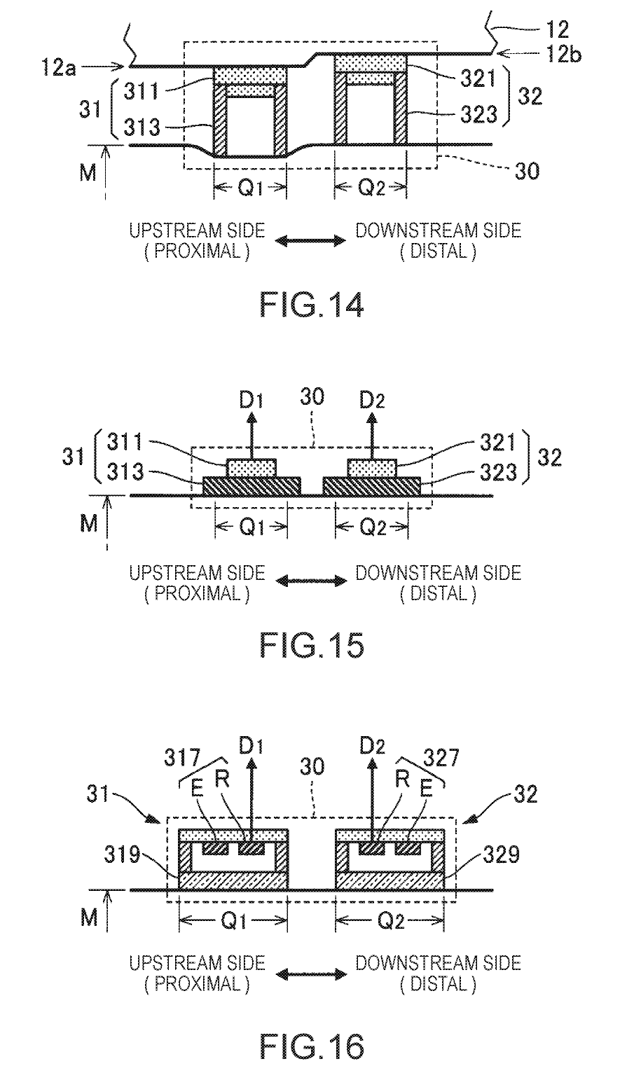

(1) The configuration for establishing the first condition and the second condition for the pressing force P1 and the pressing force P2 is not limited to the above examples. For example, as illustrated in FIG. 14, it is also possible to make the positions (heights) at which the first pulse wave detection unit 31 and the second pulse wave detection unit 32 are provided different from each other. On the housing portion 12 of the measurement apparatus 100 of FIG. 14, an installation surface 12a and an installation surface 12b are formed. The first pulse wave detection unit 31 is provided on the installation surface 12a and the second pulse wave detection unit 32 is provided on the installation surface 12b. As illustrated in FIG. 14, the installation surface 12a protrudes toward the measurement part M side as compared with the installation surface 12b. Therefore, when the housing portion 12 is attached to the measurement part M, the pressing force P1 by the first pressing portion 313 exceeds the pressing force P2 by the second pressing portion 323 (that is, the state where the first condition is satisfied). The height difference between the installation surface 12a and the installation surface 12b is set such that the pressing force P1 becomes 200 mmHg or less and the pressing force P2 is 80 mmHg or less in a state where the housing portion 12 is attached to the measurement part M.

(2) In each of the aforementioned embodiments, the pressing force P1 is set to 200 mmHg or less, but the condition for the pressing force P1 is not limited to the above examples. For example, it can be checked that the pulse wave propagation velocity V becomes an appropriate value especially in a case where the pressing force P1 is 100 mmHg or less, from FIG. 6 and FIG. 7. Considering the above tendency, a configuration in which the pressing force P1 is 100 mmHg or less is particularly preferable. That is, the second condition is that the pressing force P1 is 100 mmHg or less and the pressing force P2 is 80 mmHg or less.

(3) In each of the aforementioned embodiments, the pressing force P2 is set to 80 mmHg or less, but the condition for the pressing force P2 is not limited to the above examples. For example, the pressing force P2 can be set to the average blood pressure Bave or less of the subject. That is, the second condition is that the pressing force P1 is 200 mmHg or less and the pressing force P2 is the average blood pressure Bave. The average blood pressure Bave is expressed by the following expression using the maximum blood pressure (systolic blood pressure) Bmax and the minimum blood pressure (diastolic blood pressure) Bmin.

Bave=Bmin+(Bmax+Bmin)/3

In addition, the average blood pressure Bave can be measured using a known blood pressure monitor. It is also possible for the subject to enter its average blood pressure Bave to the measurement apparatus 100.

(4) In the second embodiment, the controller 20 controls the first driving unit 315 and the second driving unit 325, but it is also possible to provide an operator for instructing the operations of the first driving unit 315 and the second driving unit 325. The first driving unit 31 and the second driving unit 325 operate in response to the instructions for the operator from the subject.

(5) The configurations of the first pressure sensor 311 and the second pressure sensor 321 are not limited to the examples (air pressure sensor and gauge pressure sensor) in each of the aforementioned embodiments. For example, it is also possible to use a pressure sensitive element such as a piezoelectric element or a strain gauge as the first pressure sensor 311 and the second pressure sensor 321. In the configuration using the pressure sensitive element exemplified above, as illustrated in FIG. 15, the first pressing portion 313 is disposed between the first pressure sensor 311 and the measurement part M, and the second pressing portion 323 is disposed between the second pressure sensor 321 and the measurement part M. Each of the first pressing portion 313 and the second pressing portion 323 is made of a material having a thickness and hardness such that a pulse wave (for example, a fluctuation component Cb in FIG. 10) is not lost. In addition, the first pressing portion 313 and the second Dressing portion 323 can be omitted. That is, the first pressure sensor 311 is also used as the first pressing portion 313, and the second pressure sensor 321 is also used as the second pressing portion 323.

In the first embodiment, the pressing force P1 and the pressing force P2 are specified from detection results (the first detection signal D1 and the second detection signal D2) of the first pressure sensor 311 and the second pressure sensor 321, by a predetermined arithmetic expression. In the aspect of FIG. 15, the first pulse wave detection unit 31 includes a pressure sensor that detects the pressure acting on the first pressing portion 313, and the second pulse wave detection unit 32 includes a pressure sensor that detects the pressure on the second pressing portion 323. Therefore, there is an advantage that the pressing force P1 by the first pressing portion 313 can be specified from the detection result by the first pulse wave detection unit 31 and the pressing force P2 by the second pressing portion 323 can be specified from the detection result by the second pulse wave detection unit 32. In particular, since the determination processing unit 58 determines whether or not the pressing force P1 by the first pressing portion 313 and the pressing force P2 by the second pressing portion 323 are appropriate, there is an advantage that the pressing force . . . and the pressing force P2 can be easily adjusted to an appropriate range.

(6) In each of the aforementioned embodiments, the pressure sensors (the first pressure sensor 311 and the second pressure sensor 321) are used for detecting the pulse wave at the measurement part M, but a configuration for detecting a pulse wave at the measurement part M is not limited to the above examples. For example, an optical sensor that optically detects a pulse wave (photoelectric pulse wave or volume pulse wave) of the measurement part M can be used as the first pulse wave detection unit 31 and the second pulse wave detection unit 32.

Specifically, as illustrated in FIG. 16, the first pulse wave detection unit 31 includes a reflection-type first optical sensor 317 including a light emitting element E and a light receiving element R. Similarly, the second pulse wave detection unit 32 includes a reflection-type second optical sensor 327 including a light emitting element E and a light receiving element R. The light emitting element E irradiates light of a predetermined wavelength (for example, near infrared region) to the measurement part M. The light receiving element R receives the light emitted from the light emitting element E and passing through the inside of the measurement part M, and generates detection signals (D1, D2) corresponding to the received-light intensity.

As illustrated in FIG. 16, the first optical sensor 17 is covered with a first pressing portion 319 and the second optical sensor 327 is covered with a second pressing portion 329. The first pressing portion 319 and the second pressing portion 329 area light transmitting plate member. The first pressing portion 319 presses the first part Q1 of the measurement part M and the second pressing portion 329 presses the second part Q2 of the measurement part M. The pressing force P1 by the first pressing portion 319 and the pressing force P2 by the second pressing portion 329 are measured with a pressure sensor (not shown) provided separately from the first optical sensor 317 and the second optical sensor 327.

Under the configuration of FIG. 16 as well, in a state where the first pressing portion 319 presses the first part Q1 and the second pressing portion 329 presses the second part Q2, the measurement of the pulse wave propagation velocity V executed using the first detection signal D1 and the second detection signal D2 (S4). Similar to each of the above-described embodiments, the pressing force P1 by the first pressing portion 319 and the pressing force P2 by the second pressing portion 329 satisfy the first condition (P1>P2) and second condition (P1≤200 mmHg, P2≤80 mmHg). In the configuration of FIG. 16, a pressure sensor for measuring the pressing force P1 and the pressing force P2 needs to be provided separately from the first pulse wave detection unit 31 and the second pulse wave detection unit 32.

(7) In each of the aforementioned embodiments, the controller 20 and the detection device 30 are mounted on the single measurement apparatus 100, but it is also possible to realize the function of the measurement apparatus 100 by a plurality of apparatuses configured separately from each other. For example, a configuration in which the function of the controller 20 in each of the above-described embodiments is executed by a terminal device capable of communicating with the detection device 30 by radio or wire can be adopted. In addition, it is also possible to cause the terminal device communicable with the measurement apparatus 100 to execute some functions of the plurality of functions (the index measurement unit 52, the notification control unit 54, the pressing force specifying unit 56, and the determination processing unit 58) of the controller 20 of the measurement apparatus 100 according to each of the above-described embodiments. Note that the program for causing the terminal device to execute the functions (the index measurement unit 52, the notification control unit 54, the pressing force specifying unit 56, and the determination processing unit 58) exemplified in each of the above-described embodiments can be delivered the distribution device, for example, as an application program.

(8) In each of the aforementioned embodiments, the measurement of the pulse wave propagation velocity V is exemplified, but the index measurement unit 52 can calculate the time (pulse wave propagation time) Δt at which the pulse wave propagates the distance L from the first pressing portion 313 to the second pressing portion 323. The pulse wave propagation velocity V and the pulse wave propagation time At are expressed comprehensively as an index on pulse wave propagation.

The entire disclosure of Japanese Patent Application No. 2017-012243 is hereby incorporated herein by reference.

What is claimed is:

1. A measurement apparatus comprising:
a first pulse wave detection unit that detects a pulse wave at a first part pressed by a first pressing portion;
a second pulse wave detection unit that detects the pulse wave at a second part pressed by a second pressing portion, the second part being located in a direction in which a traveling wave of the pulse wave travels from the first part; and
an index measurement unit that measures an index relating to pulse wave propagation according to the pulse wave detected by the first pulse wave detection unit and the pulse wave detected by the second pulse wave detection unit,
wherein a pressing force by the first pressing portion exceeds a pressing force by the second pressing portion.

2. The measurer lent apparatus according to claim 1, wherein the pressing force by the first pressing portion is 200 mmHg or less, and the pressing force by the second pressing portion is 80 mmHg or less.

3. The measurement apparatus according to claim 2, wherein the pressing force by the first pressing portion is 100 mmHg or less.

4. The measurement apparatus according to claim 1,
wherein the first pulse wave detection unit includes a first pressure sensor that detects a pressure corresponding to displacement of the first part, and
wherein the second pulse wave detection unit includes a second pressure sensor that detects a pressure corresponding to displacement of the second part.

5. The measurement apparatus according to claim 4, further comprising:
a pressing force specifying unit that specifies the pressing force by the first pressing portion from a detection result by the first pulse wave detection unit and the pressing force by the second pressing portion from a detection result by the second pulse wave detection unit; and
a determination processing unit that determines whether or not the pressing force by the first pressing portion and the pressing force by the second pressing portion, which are specified by the pressing force specifying unit, are appropriate.

6. The measurement apparatus according to claim 1,
wherein the index measurement unit estimates a blood pressure from the index relating to the pulse wave propagation.

7. A measurement method comprising:
detecting a pulse wave at a first part and a pulse wave at a second part, in a state where a pressing force pressing the first part of a measurement part exceeds a pressing force pressing the second part located in a direction in which a traveling wave of the pulse wave travels from the first part, of the measurement part; and
measuring an index relating to pulse wave propagation according to the pulse wave at the first part and the pulse wave at the second part.
